(19) United States
(12) Patent Application Publication
(10) Pub. No.: US 2007/0292458 A1
(43) Pub. Date: Dec. 20, 2007

(54) Dentifrice Compositions Containing Zeolites

(75) Inventors: Peter William Stanier, Elworth (GB); Abraham Araya, Bebington (GB); Iam Patrick McKeown, Aigburth (GB)

Correspondence Address: JONES DAY, 222 EAST 41ST ST, NEW YORK, NY, 10017, US

(73) Assignee: Ineos Silicas Limited, Warrington Cheshire (GB)

(21) Appl. No.: 10/584,209

(22) PCT Filed: Jan. 5, 2005
(86) PCT No.: PCT/GB05/00004

§ 371 Date: Jul. 9, 2007

(30) Foreign Application Priority Data
Jan. 9, 2004 (GB) 0400408.1

(51) Int. Cl. A61K 8/25; A61Q 11/00

(52) U.S. Cl. 424/401; 424/684

(57) ABSTRACT

A dentifrice composition comprises a crystalline aluminosilicate as a cleaning agent having an average crystallite size below 0.2 μm. The aluminosilicate may be a P zeolite having a crystallite size below 0.1 μm and agglomerate sizes below 2.5 μm, even when dried to a moisture content below 20% by weight. A different crystalline aluminosilicate may also be present as a cleaning booster.

This invention relates to dentifrice compositions and in particular to dentifrice compositions comprising particular crystalline aluminosilicates as a cleaning agent.

Dentifrices commonly incorporate an abrasive material for mechanical cleaning and polishing of teeth by physical abrading of deposits and they may also include a chemical cleaning agent.

The abrasive material is primarily intended to effect mechanical removal of deposits from the surface of teeth, e.g. removal of pellicle film adhered to the tooth surface. Pellicle film is prone to discoloration and staining, e.g. by comestibles such as tea and coffee and by tars and particulates in exhaled cigarette smoke, resulting in an unsightly appearance of the teeth. While such mechanical removal is important for effective cleaning, it is vital that the abrasive used is not unduly harsh in order to minimise damage, e.g. scratching, to the teeth.

Commonly used abrasives are aluminas, calcium phosphates, calcium carbonates and amorphous silicas. Synthetic amorphous silicas are often the favoured abrasive component in dentifrices because of their efficient cleaning, compatibility with other ingredients and their physical properties such as oil absorption, pH and abrasivity. They are usually used in premium formulations, which are more expensive to produce.

Chalk is a favoured abrasive in low cost dentifrices and can provide an affordable fluoride-containing toothpaste where such a low cost product is desired. The chalk is generally available as fine ground natural chalk or precipitated calcium carbonate and is usually used at incorporation levels of 30 to 50% by weight.

Crystalline aluminosilicates (zeolites) have been used as cleaning agents in dentifrice compositions. They possess a mechanical cleaning action (abrasivity) and are also known to bind calcium ions. Desirably, a dental cleaning agent combines relatively good cleaning with minimal abrasion of dentine. It has been found that most available zeolites are generally too abrasive to provide adequate cleaning without unacceptable abrasion, especially because higher incorporation levels are required to give good cleaning.

GB 2082454 to W R Grace uses a synthetic zeolite polishing and cleaning agent having an average crystal size of less than 1 μm. It is preferred that the zeolite is a filter cake to avoid the agglomeration of crystals into larger sizes that occurs on drying.

Surprisingly, it has now been found that a specific aluminosilicate can be produced and used in the dry form with very small crystallite size, which has lower levels of abrasivity and good cleaning properties when incorporated in a dentifrice composition. It is particularly useful when used at higher loadings.

According to the invention a dentifrice composition comprises a crystalline aluminosilicate having an average crystallite size below 0.2 μm.

Crystalline aluminosilicates useful in the dentifrice composition of this invention can be represented by the formula:
M_{2/n}O·Al₂O₃·xSiO₂·yH₂O
wherein M represents a metal moiety, said metal having a valency of n, x indicates the molar ratio of silica to alumina and y indicates the ratio of molecules of water to alumina.

The structure and characteristics of many crystalline aluminosilicates (zeolites) are described in the standard work “Zeolite Molecular Sieves” by Donald W. Breck, published by Robert E. Krieger Publishing Company. Usually, the value of x in the above empirical formula is in the range 1.5 to 10. The value of y, which represents the amount of water contained in the voids of the zeolite, can vary widely. In anhydrous material y=0 and, in fully hydrated zeolites, y is typically up to 5.

Zeolites useful in this invention may be based on naturally-occurring or synthetic aluminosilicates but a preferred form of zeolite has the structure known as zeolite P. Particularly preferred forms of zeolite are those disclosed in EP-A-0 384 070, EP-A-0 565 364, EP-A-0 697 010, EP-A-0 742 780, WO-A-96/14270, WO-A-96/34828 and WO-A-97/06102, the entire contents of which are incorporated herein by this reference. The zeolite P described in EP-A-0 384 070 has the empirical formula given above in which M represents an alkali metal and x has a value up to 2.66, preferably in the range 1.8 to 2.66, and has a structure which is particularly useful in the present invention. More preferably, x has a value in the range 1.8 to 2.4. The zeolite P disclosed in the above patent literature is readily amenable to being produced with crystallite sizes well below 0.2 μm and agglomerate sizes below 2.5 μm, even when dried to a moisture content below 20% by weight. This contrasts with other zeolites which, on drying, tend to agglomerate to large weight mean particle sizes.

Usually, the preferred form of zeolite P is one in which M in the above formula consists of alkali metal ions. However, suitable forms of zeolite P include those wherein a proportion of the alkali metal moieties M have been exchanged for multivalent metal moieties. Partially exchanged zeolites are particularly useful when it is desired to control the pH of the abrasive system.

The pH of the aluminosilicate used in the composition of the invention, particularly when not partially exchanged as discussed above, is usually in excess of 10. Where the aluminosilicate present in the composition is one which has undergone such ion exchange, its pH will usually be no greater than 10. The average crystallite size of the crystalline aluminosilicate, measured using the test described hereinafter is preferably between 0.01 and 0.1 μm (typically less than 0.1 μm) and, more preferably between 0.02 and 0.08 μm or less

The crystalline aluminosilicate should have a Radioactive Dentine Abrasion (RDA) which is relatively low and is preferably less than 120, more preferably less than 100. Its RDA will usually be in excess of 30. The RDA values which characterise the aluminosilicate used in the system of this invention are measured using an aqueous slurry of the aluminosilicate as defined in the test described hereinafter.

If however the RDA were measured on the complete dentifrice composition i.e. including any optional components as defined hereinafter, the RDA values obtained may be significantly different. For example the RDA would be in the range 25-200, preferably 30-180, more preferably 50-150.

Additionally, preferred aluminosilicates produce minimal scratching on dental surfaces when used. Scratching can be assessed using the PAV test described hereinafter and preferred aluminosilicates have a PAV of 4 to 11, preferably 4 to 9 and more preferably 4 to 7.

The aluminosilicate has a calcium binding capacity, as hereinafter defined, of at least 100 mg CaO per gram of anhydrous aluminosilicate, preferably at least 130 mg CaO per gram of anhydrous aluminosilicate and most preferably at least 150 mg CaO per gram of anhydrous aluminosilicate.

The aluminosilicate preferably has an oil absorption greater than 40 cm³/100 g and preferably in the range 40 to 100 cm³/100 g.

The aluminosilicate preferably has a weight mean particle size as measured by Malvern Mastersizer®, of at least 0.5 μm, more usually at least 1.0 μm, e.g. at least 1.8 μm. The aluminosilicate preferably has a weight mean particle size as measured by Malvern Mastersizer®, of at most 10.0 μm, more usually at most 5.0 μm e.g. at most 3.0 μm. A most preferred range for the aluminosilicate is from 2.0 to 2.5 μm.

The crystalline aluminosilicate can be the only abrasive/cleaning agent present in the dentifrice compositions of the invention or additional abrasive/cleaning agents may also be present. One such additional abrasive/cleaning agent is a different crystalline aluminosilicate, e.g. an A, X or Y type zeolite, which acts as a cleaning booster (hereinafter referred to as “booster zeolite”). When present, the amount of booster zeolite present will usually be less than that of the zeolite referred to hereinbefore (the “principal” zeolite).

This booster zeolite preferably has an RDA in the range 100 to 300 and more preferably in the range 100 to 250. The PAV of the booster zeolite is preferably in the range 9 to 25 and more preferably in the range 9 to 20. The values for both the RDA and the PAV of the booster zeolite will be greater than those for the principal zeolite. The preferred oil absorption of the booster zeolite is in the range 30 to 100 cm³/100 g and more preferably 30 to 50 cm³/100 g. The weight mean particle size of the booster zeolite is preferably in the range 2.0 to 5.0 μm. The booster zeolite typically has an average crystallite size above 0.2 μm and preferably above 1.0 μm.

The proportions of crystalline aluminosilicate and booster zeolite present in the dental abrasive system of the invention can be varied to provide optimum cleaning with controlled abrasion. Generally, the proportion of crystalline aluminosilicate to booster zeolite by weight is in the range 40:1 to 1:1. Preferably, the ratio is in the range 9:1 to 3:2 and most preferably within the range 4:1 to 7:3.

The amount of crystalline aluminosilicate present in the dentifrice composition of the invention (not including any booster zeolite present) is preferably in the range 10 to 50 percent by weight of the dentifrice composition. When the particular aluminosilicate is used as the sole abrasive/cleaning agent, it is preferably present in the range 25 to 50 percent by weight, more preferably in the range 35 to 50 percent by weight and most preferably in the range 35 to 45 percent by weight of the dentifrice composition. When the particular aluminosilicate is used in association with a booster zeolite, it is more preferably present in the range 10 to 40 percent by weight and most preferably in the range 25 to 35 percent by weight of the dentifrice composition.

When present, the booster zeolite is preferably present in an amount of up to 15 percent by weight of the dentifrice composition, more preferably in an amount in the range 1 to 10 percent by weight and most preferably in an amount in the range 1 to 6 percent by weight of the dentifrice composition.

The dentifrice compositions of the invention are capable of providing effective cleaning, with controlled levels of abrasion, as measured by RDA, and at lower cost than silica formulations with similar performance.

The cleaning ability of a composition can be assessed by the test known as the FT₁₀₀ Cleaning test, details of which are given below. The abrasive systems are tested in the dentifrice composition. Preferred dentifrice compositions of this invention have a FT₁₀₀ Cleaning value of at least 50 percent, more preferably at least 60 percent and most preferably above 70 percent.

The dentifrice compositions of the invention can be in any form suitable as a dentifrice composition, such as a paste, gel, cream, liquid, chewing gum or powder.

When the dentifrice composition is a toothpaste, cream or liquid comprising the aluminosilicate in an orally acceptable vehicle, the orally acceptable vehicle typically contains at least one humectant, for example a polyol such as glycerol, sorbitol syrup, polypropylene glycol, polyethylene glycol, lactitol, xylitol or hydrogenated corn syrup. The total amount of humectant is usually in the range from about 10 to about 85 percent by weight of the composition, preferably in the range from about 10 to about 50 percent by weight of the composition and most preferably in the range about 10 to about 40 percent by weight of the composition.

The term “orally acceptable carrier” means a suitable vehicle which can be used to apply the present compositions to the oral cavity in a safe and effective manner.

Water is usually present as a component of the dentifrice compositions of the invention normally in an amount of from about 1 to about 90 percent by weight, preferably from about 10 to about 60 percent, more preferably from about 15 to about 30 percent by weight.

The orally acceptable vehicle may optionally comprise one or more surfactants, sweetening agent, flavouring agent, anticaries agent (in addition to a fluoride ion source), anti-plaque agent, anti-bacterial agent such as triclosan or cetyl pyridinium chloride, tooth desensitizing agent such as potassium nitrate or strontium fluoride, colouring agents and pigment.

The surfactant may be selected from anionic, non-ionic and zwitterionic surfactants, and mixtures thereof, all being suitable for oral use. The amount of surfactant present in the composition of the invention is typically from about 0.1 to about 3 percent by weight (based upon 100 percent surfactant activity).

Suitable anionic surfactants may include soaps, alkyl sulphates (e.g. the water-soluble salts of alkyl sulphates having from 10 to 18 carbon atoms in the alkyl moiety, such as sodium lauryl sulphate), alkyl ether sulphates, alkaryl sulphonates, alkanoyl isethionates, alkanoyl taurates, alkyl succinates, alkyl sulphosuccinates, N-alkoyl sarcosinates, alkyl phosphates, alkyl ether phosphates, alkyl ether carboxylates and alpha-olefin sulphonates, especially their sodium, magnesium, ammonium and mono-, di- and tri-ethanolamine salts. The alkyl and acyl groups generally contain from 8 to 18 carbon atoms and may be saturated. The alkyl ether sulphates, alkyl ether phosphates and alkyl ether carboxylates may contain from one to 10 ethylene oxide or propylene oxide units per molecule, and preferably 2 to 3 ethylene oxide units per molecule. Examples of preferred anionic surfactants include sodium lauryl sulphate, sodium dodecylbenzene sulphonate, sodium lauroyl sarcosinate and sodium coconut monoglyceride sulphonate.

Non-ionic surfactants which may be suitable for use in the dentifrice composition of the invention include sorbitan and polyglycerol esters of fatty acids, as well as ethylene oxide/propylene oxide block copolymers.

Suitable zwitterionic surfactants include betaines such as cocamidopropyl betaine and sulphobetaines.

The dentifrice compositions of the present invention preferably include one or more thickening agents and/or suspending agents in order to give the composition the desired physical properties (e.g. whether a paste, cream or a liquid) and in order that the aluminosilicate remains stably dispersed throughout the composition.

A particularly preferred means for thickening the dentifrice compositions of the invention is by the inclusion of a thickening silica, usually in conjunction with a polymer suspending or thickening agent. Suitable well-known polymer suspending or thickening agents, which may be used alone or in conjunction with a thickening silica, include polyacrylic acid, copolymers and cross-linked polymers of acrylic acid, copolymers of acrylic acid with a hydrophobic monomer, copolymers of carboxylic acid-containing monomers and acrylic esters, cross-linked copolymers of acrylic acid and acrylate esters, esters of ethylene glycol or esters of polyethylene glycol (e.g. fatty acid esters thereof), heteropolysaccharide gums such as xanthan and guar gums, and cellulose derivatives such as sodium carboxymethyl cellulose. Particularly suitable suspending or thickening agents are xanthan gum and sodium carboxymethyl cellulose. These thickeners (which may be used singly or as mixtures of two or more of the above materials) may be present in the composition in a total amount of from about 0.1 to about 5 percent by weight. When used with silica thickening agents they are preferably present in an amount in the range 0.1 to 5.0 percent by weight. Silica thickening agents, such as the silica sold under the trade name Sorbosil TC15 by INEOS Sllicas Limited, Warrington, UK, when present, comprise from about 0.1 to about 15 percent by weight, preferably about 1 to about 10 percent by weight of the composition.

One or more other components that are conventionally found in a dentifrice composition may be present in the dentifrice composition and include the following; flavouring substances such as peppermint, spearmint; artificial sweeteners; perfume or breath freshening substances; pearlescing agents; peroxy compounds such as hydrogen peroxide or peracetic acid; opacifiers; pigments and colourings; preservatives; moisturizing agents; fluoride-containing compounds; anti-caries and anti-plaque agents; anti-tartar agents; anti-hypersensitivity agents; therapeutic agents such as zinc citrate, Triclosan (ex Ciba Geigy); proteins; enzymes; salts; baking soda and pH adjusting agents.

If an aqueous orally acceptable vehicle is employed, the dentifrice composition suitably contains from about 10 to about 80 wt % humectant such as sorbitol, glycerin, polyethylene glycol or xylitol; from about 0.25 to about 5 wt % detergent; from 0 to about 2 wt % sweetener, from 0 to about 2 wt % flavouring agents; together with water and an effective amount of binding and thickening agents, such as from about 0.1 to about 15 wt %, to provide the composition, e.g. a toothpaste, of the invention with the desired stability and flow characteristics.

Preferably the pH of the dentifrice composition of the present invention is from about 6 to 10.5, more preferably from about 7 to about 9.5. Typically the composition may contain sodium hydroxide, e.g. up to 1.0 wt. % or more, to provide a suitable pH.

The orally acceptable carrier may also include a fluoride ion source as protection against demineralisation by bacteria (caries) and/or acidic components of the diet (erosion). The fluoride ion source may be provided by any of the compounds conventionally used in toothpastes for these purposes, e.g. sodium fluoride, alkali metal monofluorophosphate, stannous fluoride and the like, with an alkali metal monofluorophosphate such as sodium monofluorophosphate being preferred. The fluoride ion source serves in a known manner for caries protection. Preferably, the fluoride ion source will be used in an amount to provide a safe yet effective amount to provide an anti-caries and anti-erosion benefit such as an amount sufficient to provide from about 25 ppm to about 3500 ppm, preferably about 1100 ppm, as fluoride ion. For example the formulation may contain 0.1-0.5 wt % of an alkali metal fluoride such as sodium fluoride.

Dentifrice compositions in accordance with the invention may be made by conventional methods for preparing such compositions. Pastes and creams may be prepared by conventional techniques, for example, using high shear mixing systems under vacuum.

The tests used to characterise the abrasive/cleaning agents used in the dentifrice compositions of this invention are as follows.

Radioactive Dentine Abrasion Test (RDA)

The procedure follows the method for assessment of dentifrice abrasivity recommended by the American Dental Association (Journal of Dental Research 55(4) 563, 1976). In this procedure, extracted human teeth are irradiated with a neutron flux and subjected to a standard brushing regime. The radioactive phosphorus 32 removed from the dentin in the roots is used as the index of the abrasion of the dentifrice tested. A reference slurry containing 10 g of calcium pyrophosphate in 50 cm³ of 0.5% aqueous solution of sodium carboxymethyl cellulose is also measured and the RDA of this mixture is arbitrarily taken as 100. In order to measure a powder RDA for the crystalline aluminosilicate suspension of 10.0 g of the aluminosilicate in 50 cm³ of 0.5% aqueous solution of sodium carboxymethyl cellulose is prepared and the suspension is submitted to the same brushing regime. In order to measure an RDA value for a dentifrice composition of the invention a test slurry is prepared from 25 g dentifrice composition and 40 g water and this slurry is submitted to the same brushing regime.

Plastics Abrasion Value (PAV)

This test is based upon a toothbrush head brushing a Perspex® plate in contact with a suspension of aluminosilicate in a sorbitol/glycerol mixture. Perspex® has a similar hardness to dentine. Therefore, a substance which produces scratches on Perspex® is likely to produce a similar amount of scratching on dentine. Normally the slurry concentration is as follows:

| | |
|---|---|
| Aluminosilicate | 2.5 g |
| Glycerol | 10.0 g |
| Sorbitol Syrup* | 23.0 g |

*Syrup contains 70% sorbitol/30% water

All components are weighed into a beaker and dispersed for 2 minutes at 1500 rpm using a simple stirrer. A 110 mm×55 mm×3 mm sheet of standard PERSPEX clear cast acrylic sheet, grade 000, manufactured by Lucite International UK Ltd, PO Box 34, Darwen, Lancashire, UK, is used for the test.

The test is carried out using a modified Wet Scrub Abrasion Tester produced by Sheen Instruments. The modification is to change the holder so that a toothbrush can be used in place of a paintbrush. In addition, a weight of 400 g is attached to the brush assembly, which weighs 145 g, to force the brush onto the PERSPEX sheet. The toothbrush has a multi-tufted, flat trim nylon head with round ended filaments and medium texture, for example, the well-known Professional Mentadent P gum health design, or an equivalent toothbrush.

A galvanometer is calibrated using a 45° Plaspec gloss head detector and a standard (50% gloss) reflecting plate. The galvanometer reading is adjusted to a value of 50 under these conditions. The reading of the fresh PERSPEX sheet is then carried out using the same reflectance arrangement. The fresh piece of PERSPEX sheet is then fitted into a holder. 2 cm³ of the dispersed aluminosilicate, sufficient to lubricate fully the brushing stroke, is placed on the sheet and the brush head is lowered onto the sheet. The machine is switched on and the sheet is subjected to 300 strokes of the weighted brush head. The sheet is removed from the holder and all the suspension is washed off. It is then dried and its gloss value is determined again. The abrasion value is the difference between the unabraded gloss value and the gloss value after abrasion. This test procedure, when applied to known abrasives, gave the following typical values.

| | PAV |
|---|---|
| Calcium carbonate (15 μm) | 32 |
| Silica xerogel (10 μm) prepared according to GB 1 262 292 | 25 |
| Alumina trihydrate (Gibbsite) (15 μm) | 16 |
| Calcium pyrophosphate (10 μm) | 14 |
| Dicalcium phosphate dihydrate (15 μm) | 7 |

Oil Absorption

The oil absorption is determined by the ASTM spatula rub-out method (American Society of Test Material Standards D 281). The test is based on the principle of mixing linseed oil with the aluminosilicate by rubbing with a spatula on a smooth surface until a stiff putty-like paste is formed which will not break or separate when it is cut with a spatula. The oil absorption is then calculated from the volume of oil (V cm³) used to achieve this condition and the weight, W, in grams, of aluminosilicate by means of the equation:
Oil absorption=(V×100)/W, i.e. expressed in terms of cm³ oil/100 g aluminosilicate.
Weight Mean Particle Size by Malvern Mastersizer®

The weight mean particle size of the aluminosilicate is determined using a Malvern Mastersizer® model S, with a 300 RF lens and MS17 sample presentation unit. This instrument, made by Malvern Instruments, Malvern, Worcestershire, uses the principle of Fraunhofer diffraction, utilising a low power He/Ne laser. Before measurement, the sample is dispersed ultrasonically in water for 30 seconds to form an aqueous suspension. The Malvern Mastersizer® measures the weight particle size distribution of the aluminosilicate. The weight mean particle size (d₅₀D) or 50 percentile and the percentage of material below any specified size are easily obtained from the data generated by the instrument.

Average Crystallite Size of Aluminosilicate

The average crystallite size is determined from photographs made in a scanning electron microscope. The crystalline aluminosilicate is dried to a water content of about 1 to 3 weight percent and the agglomerates are broken up with a pestle and mortar. From the photographs, a sufficient number of crystals, e.g. 100, is counted and their size measured to determine a statistically significant average (arithmetical mean) size.

Calcium Binding Capacity of Aluminosilicate

The aluminosilicate is first equilibrated to constant weight over saturated sodium chloride solution and the water content is measured. An amount is dispersed in 1 cm³ water in an amount corresponding to 1 g dm⁻³ (dry weight) and the resulting dispersion is injected into a stirred solution of total volume 54.923 cm³, consisting of 0.01M NaCl solution (50 cm³) and 0.05M CaCl₂ (3.923 cm³). This corresponds to a concentration of 200 mg of CaO per dm³, i.e. just greater than the theoretical maximum amount (197 mg) that can be taken up by an aluminosilicate of Si:Al ratio 1.00. The dispersion is vigorously stirred at a temperature of 25° C. for 15 minutes, after which time the Ca²⁺ ion concentration is determined using a calcium electrode. The Ca²⁺ ion concentration measured is subtracted from the initial concentration to give the effective calcium binding capacity of the aluminosilicate sample.

FT₁₀₀ Cleaning Test

The test is fully described in “Dental stain prevention by abrasive toothpastes: A new in vitro test and its correlation with clinical observations”, P. L. Dawson et al., J. Cosmet. Sci., 49, 275-283 (1998).

The abrasive system to be tested is incorporated into the following Dental Formulation 1.

Dental Formulation 1

| INGREDIENT | % by wt. |
|---|---|
| Sorbitol, 70% Soln. | 26.00 |
| Glycerol, 98% min. solution | 10.00 |
| Polyethylene glycol (PEG 6) | 3.00 |
| Crystalline aluminosilicate | A |
| Booster Zeolite | B |
| Silica thickener | C |
| Sodium lauryl sulphate | 1.15 |
| Titanium Dioxide | 1.45 |
| Xanthan gum | 0.7 |
| Saccharin | 0.23 |
| Flavour | 1 |
| NaF | 0.24 |
| Deionised water | To 100 |
| Total | 100.00 |

The quantities A and B are determined by the abrasive system under test. The quantity of thickening silica (“C”) is adjusted to ensure that the cohesion of the paste, as measured by the toothpaste cohesion test defined hereinafter is in the range 150 to 430 g.

Substrate

A substrate consisting of highly polished 17 mm sintered, pure hydroxyapatite (HAP) discs is prepared. The discs are polished using a Buehler rotary grinder and P600 wet paper, followed by P1200 lapping paper to give a mirror-like finish to simulate enamel tooth surface. The whiteness of the discs (using the CIE 1976 L*a*b* system) before cleaning, L* (clean), is then measured using a Minolta Chroma-meter CR200, which has been calibrated against a standard calibration tile.

Staining

A fresh staining solution is prepared by mixing 50 g of a 0.5% by weight solution of tannic acid and 50 g of a 0.5% by weight solution of ammonium ferric sulphate to form a fresh colloidal iron (III) tannic acid complex (“ferric tannate”), which has a dark colour. The fresh mixture is painted on to the HAP discs using a fine squirrel-hair brush and gently dried with a warm hairdryer. A sufficient number of coats of staining solution are applied in order to produce a darkness measurement of L*=50+/−5 as determined using a Minolta Chroma-meter CR200. This value is designated L* (soiled).

Toothpaste Slurry Preparation

A diluent is prepared, which consists of:

| | % by weight |
|---|---|
| Sodium carboxymethyl cellulose (SCMC 7M) | 0.5 |
| Glycerol | 5.0 |
| Formalin | 0.1 |
| Demineralised Water | 94.4 |

The toothpaste under test is weighed into a plastic beaker and mixed with diluent and demineralised water in the following proportions by weight:

Toothpaste 33.3%; Diluent 33.3%; Water 33.3%

to produce a 100 g toothpaste slurry preparation, which is mixed for one minute with a high shear Heidolph mixer at 4000 r.p.m.

The toothpaste slurry should be prepared immediately prior to carrying out the test to avoid any chances of settlement.

Brushing

The stained HAP discs are then mounted horizontally in the bottom of a trough containing the toothpaste slurry under test and 263 g weighted toothbrush heads are oscillated over the disc surfaces using a mechanical scrubbing machine (modified Martindale abrasion tester). An oscillation rate of 150 cycles per minute is used. The toothbrush heads are 34-tuft flat-trim 0.2 mm bristle nylon heads and are weighted via weights loaded onto vertical spindles mounted in linear ball bearings. For the FT₁₀₀ test soil removal after 100 oscillations is monitored. The whiteness of the HAP discs after cleaning, L* (cleaned) is measured using a Minolta Chroma-meter CR200. The FT₁₀₀ Cleaning is defined as the % FT₁₀₀ Removal where

$$\% {FT}_{100} Removal = \frac{L * ( cleaned ) - L * ( soiled )}{L * ( clean ) - L * ( soiled )} \times 100$$

Toothpaste Cohesion

The cohesion of a toothpaste is a good measure of the “stand-up” properties of the ribbon when it has been extruded from a toothpaste tube onto a toothbrush. Higher cohesion values indicate firmer toothpaste ribbons, whereas low cohesion numbers are obtained from low viscosity, poorly structured toothpastes, which quickly sag into the bristles of the brush. It is generally required that a dentifrice has a cohesion within the range of 150-430 g to provide a good quality, extrudable ribbon, which does not sag and is not too firm.

The basic principle of the test is to measure the weight in grams required to pull two parallel plates apart, which have a specific layer of toothpaste sandwiched between them. The purpose built equipment consists of:

1) A spring balance in which the spring can be extended from 0-430 g in 100 mm of length. The spring has a calibration scale of zero to 430 g in 10 g intervals and can be adjusted to zero at the start of the test.

2) A motor driven ratchet, which is attached to the bottom plate and can be used to apply a constant, uniform, smooth vertical pull on the bottom plate of 5 cm per minute.

3) An upper polished chrome circular plate of 64 mm diameter, which has a hook on the upperside that can be attached to the spring balance. The polished plate has three small identical spacer pieces of polished chrome on the underside of the plate, as an integral part of the plate. These protrude to a depth of 4 mm, which determines the toothpaste film thickness when the equipment is assembled to carry out the test.

4) A lower polished chrome circular plate of 76 mm diameter, which is attached underneath to a motor driven ratchet. Two short pegs are located on the top of the plate so that the top plate can be positioned on the bottom plate concentrically from the centres.

5) A metal framework which allows the top plate to be situated concentrically above the bottom plate and the bottom plate to be adjusted so that the plate is approximately horizontal (achieved through the use of levelling feet on the base of the equipment).

15-20 g of toothpaste is evenly distributed onto the underside of the upper plate and the plate is carefully positioned onto the top of the bottom plate, using the two short pegs to locate the edge of the top plate. The top plate is firmly pressed down onto the bottom plate, until all three spacers have made contact with the bottom plate. Excess toothpaste, which has been squeezed out from between the two plates is then removed with a spatula, such that no toothpaste extends beyond the diameter of the top plate. The upper plate is then connected to the spring balance and the scale set to zero grams. The equipment is then switched on to allow the motor driven ratchet to lower the bottom plate. The spring is gradually extended and the highest observed weight is noted, as the two parallel plates sandwiched with toothpaste are eventually pulled apart. This is the toothpaste cohesion recorded in grams.

The invention is illustrated by the following non-limiting examples.

### EXAMPLES

In order to demonstrate the use of the invention, the aforementioned Dental Formulation 1 was used as a base formulation in which particle components A, B, and C were varied according to the following examples:

### Example 1

40% by weight Doucil A24 Zeolite was used as the crystalline aluminosilicate abrasive, A, in dental formulation 1. There was no booster zeolite, B, or thickening silica, C, in this example. The properties of the cleansing particles used are given in Table 1.

The properties of the resultant toothpaste are given in Table 2.

### Example 2

14% by weight of Doucil A24 Zeolite was used as the crystalline aluminosilicate abrasive, A, and 6% by weight Doucil 4A Zeolite was used as the booster zeolite, B, in dental formulation 1, together with 7% by weight thickening silica, C. The properties of the cleansing particles used are also given in Table 1.

The properties of the resultant toothpaste are also given in Table 2

TABLE 1
Zeolite Powder Properties

| | | | | | | | Calcium |
|---|---|---|---|---|---|---|---|
| | | | Oil | Weight mean | Average | | binding |
| | Powder | | absorption | particle size | Crystallite | | capacity |
| Particle type | RDA | PAV | (cm³/100 g) | (μm) | size (μm) | pH | mg CaO/g |
| Doucil A24 Zeolite | 82 | 8 | 58 | 2.25 | 0.06 | 11.4 | 160 |
| Doucil 4A Zeolite | 157 | 11.3 | 40 | 3.93 | 1.0 | 11.3 | 145 |

TABLE 2
Toothpaste Properties

| Example | Toothpaste | Toothpaste | Toothpaste |
|---|---|---|---|
| No | RDA | FT₁₀₀ Cleaning | Cohesion(g) |
| 1 | 128 | 61.1 | 225 |
| 2 | 77 | 72.8 | 255 |

1. A dentifrice composition comprising a crystalline aluminosilicate having an average size below 0.2 μm, in which the aluminosilicate is a zeolite having the formula M_{2/n}O—Al₂O₃-xSiO₂-yH₂O wherein M represents a metal moiety having a valency of n, x indicates the molar ratio of silica to alumina and V indicates the ratio of molecules of water to alumina wherein the zeolite is a P zeolite having a weight mean particle size of at most 10.0 μm.

2.-3. (canceled)

4. A dentifrice composition as claimed in claim 1 in which x has a value up to 2.66.

5. A dentifrice composition as claimed in claim 1 in which x has a value in the range 1.8 to 2.66.

6. A dentifrice composition as claimed in claim 1 in which x has a value in the range 1.8 to 2.4.

7. A dentifrice composition as claimed in claim 1 in which the aluminosilicate has a moisture content below 20% by weight.

8. A dentifrice composition as claimed in claim 1 in which the average crystallite size of the crystalline aluminosilicate is between 0.01 and 0.2 μm.

9. A dentifrice composition as claimed in claim 1 in which the average crystallite size of the crystalline aluminosilicate is less than 0.1 μm.

10. A dentifrice composition as claimed in claim 1 in which the average crystallite size of the crystalline aluminosilicate is up to 0.08 μm.

11. A dentifrice composition as claimed in claim 1 in which the RDA of the crystalline aluminosilicate is not greater than 120.

12. A dentifrice composition as claimed in claim 1 in which the aluminosilicate has a PAV of up to 11, preferably up to 9.

13. A dentifrice composition as claimed in claim 1 in which the aluminosilicate has a calcium binding capacity of at least 130 mg CaO per gram of anhydrous aluminosilicate.

14. A dentifrice composition as claimed in claim 1 in which the aluminosilicate has an oil absorption of at least 40 cm³/100 g.

15. A dentifrice composition as claimed in claim 1 in which the aluminosilicate has a weight mean particle size of at least 0.5 μm.

16. (canceled)

17. A dentifrice composition as claimed in claim 1 in which the aluminosilicate has a weight mean particle size in range 2.0 to 2.5 μm.

18. A dental composition as claimed in claim 1 in which the crystalline aluminosilicate is derived from a zeolite P with the formula M_{2/n}O—Al₂O₃-xSiO₂-yH₂O where M is an alkali metal and in which at least a proportion of the alkali metal M has been exchanged for one or more other metal moieties.

19. A dental composition as claimed in claim 1 containing at least one additional abrasive/cleaning agent.

20. A dentifrice composition as claimed in claim 19 in which the additional agent comprises a different crystalline aluminosilicate, which acts as a cleaning booster.

21. A dentifrice composition as claimed in claim 19 in which the cleaning booster has an RDA in the range 100 to 300.

22. A dentifrice composition as claimed in claim 19 in which the cleaning booster has an RDA in the range 100 to 250.

23. A dentifrice composition as claimed in claim 19 in which the RDA of the first-mentioned aluminosilicate is less than 100.

24. A dentifrice composition as claimed in claim 19 in which the cleaning booster has a PAV of in the range 9 to 25.

25. A dentifrice composition as claimed in claim 19 in which the PAV of the first-mentioned aluminosilicate is less than 9.

26. A dentifrice composition as claimed in claim 19 in which the oil absorption of the cleaning booster is in the range 30 to 100 cm³/100 g.

27. A dentifrice composition as claimed in claim 19 in which the weight mean particle size of the cleaning booster is in the range 2.0 to 5.0 μm.

28. A dentifrice composition as claimed in claim 19 in which the cleaning booster has an average crystallite size above 0.2 μm.

29. A dentifrice composition as claimed in claim 19 in which the cleaning booster has an average crystallite size above 1.0 μm.

30. A dentifrice composition as claimed in claim 19 in which the proportions of the first-mentioned aluminosilicate and the cleaning booster present by weight are in the range 40:1 to 1:1

31. A dentifrice composition as claimed in claim 19 in which the proportions of the first-mentioned aluminosilicate and the cleaning booster present by weight are in the range 9:1 to 3:2.

32. A dentifrice composition as claimed in claim 19 in which the proportions of the first-mentioned aluminosilicate and the cleaning booster present by weight are in the range 4:1 to 7:3.

33. A dentifrice composition as claimed in claim 1 in which the amount of crystalline aluminosilicate present (not including any cleaning booster present) is in the range 10 to 50 percent by weight of the dentifrice composition.

34. A dentifrice composition as claimed in claim 1 in which the amount of crystalline aluminosilicate present (not including any cleaning booster present) is in the range 25 to 50 percent by weight of the dentifrice composition.

35. A dentifrice composition as claimed in claim 19 in which the amount of the first-mentioned aluminosilicate present is in the range 10 to 40 percent by weight of the dentifrice composition.

36. A dentifrice composition as claimed in claim 19 in which the amount of the first-mentioned aluminosilicate present is in the range 25 to 35 percent by weight of the dentifrice composition.

37. A dentifrice composition as claimed in claim 19 in which the cleaning booster is present in an amount of up to 15 percent by weight of the dentifrice composition.

38. A dentifrice composition as claimed in claim 19 in which the cleaning booster is present in an amount in the range 1 to 10 percent by weight.

39. A dentifrice composition as claimed in claim 1 having an FT₁₀₀ Cleaning value of at least 50 percent.

40. A dentifrice composition as claimed in claim 1 having an FT₁₀₀ Cleaning value of at least 70 percent.
